(19) United States
(12) Patent Application Publication
(10) Pub. No.: US 2006/0235299 A1
(43) Pub. Date: Oct. 19, 2006

(54) Apparatus and method for intravascular imaging

(76) Inventor: Michael A. Martinelli, Winchester, MA (US)

Correspondence Address: Donald E. Mahoney, 57 Eisenhower Circle, Wellesley, MA, 02482, US

(21) Appl. No.: 11/400,996

(22) Filed: Apr. 10, 2006

Related U.S. Application Data
(60) Provisional application No. 60/671,008, filed on Apr. 13, 2005.

(51) Int. Cl. A61M 25/00

(52) U.S. Cl. 600/434; 600/462

(57) ABSTRACT

A method and apparatus for intravascular imaging utilizes a rotating magnetic field generated outside of the patient's body to cause a substantially synchronous rotation of an ultrasonic signal inside the patient's body.

### CROSS-REFERENCE TO RELATED APPLICATIONS

THIS APPLICATION CLAIMS THE BENEFIT OF U.S. PROVISIONAL APPLICATION NO. 60/671,008, FILED Apr. 13, 2005

### FEDERALLY SPONSORED RESEARCH

Not Applicable

### BACKGROUND OF THE INVENTION

1. Field of the Invention

The present invention relates to imaging guidewires and catheters, more particularly, to intravascular imaging guidewires and catheters that can scan an ultrasonic signal against tissues surrounding the guidewire or catheter by utilizing a rotating magnetic field applied from outside a patient's body to rotate a permanent magnet disposed within the guidewire or catheter.

2. Description of Related Art

Ultrasonic imaging of tissue surrounding a vascular cavity has long been a tool for determining the condition of such tissue. Apparatus for introducing ultrasonic signals into a desired location in a vascular cavity have included imaging guidewires and catheters adapted to slide along a guidewire. The path in the vascular cavity along which the imaging guidewire or catheter travels can often be tortuous causing difficulties for the various mechanical or electrical devices used for causing ultrasonic signals to scan surrounding tissue. Examples of such imaging guidewires or catheters are described in U.S. Pat. No. 5,779,643 (Lum, et. al.) U.S. Pat No. 4,794,931 (Yock), U.S. Pat. No. 5,000,185 (Yock), U.S. Pat. No. 5,240,003 (Lance, et. al.), U.S. Pat. No. 5,176,141 (Bom, et. al.), U.S. Pat. No. 5,271,402 (Yeung and Dias), U.S. Pat. No. 5,284,148 (Dias and Melton).

One problem encountered by known ultrasonic probes is failure of a drive cable operated by a motor located outside of the patient's body and connected to a transducer or reflector disposed within the probe. Oftentimes the drive cable is unable to provide uniform rotation of the transducer or reflector, causing artifacts in the ultrasound image of tissue surrounding the probe. Sometimes rapid and repetitive rotations of the drive cable will result in cable failure. What is needed is an ultrasonic probe that can scan surrounding body tissue without the need of a drive cable connected to a remotely located motor or small motor located within the ultrasonic probe.

### SUMMARY OF THE INVENTION

According to the present invention an apparatus and method for imaging tissues from inside a patient's body comprising a tubular housing having a portion substantially transparent to ultrasonic signals; a permanently magnetized slug having at least one beveled end with the slug being rotatively disposed within the tubular body; means for generating an ultrasonic signal and transmitting this signal toward the beveled end of the magnetized slug, and means for generating a rotating magnetic field from outside of the patient's body for rotating the slug inside the patient's body.

### BRIEF DESCRIPTION OF THE DRAWINGS

The invention is better understood by reading the following detailed description with reference to the accompanying figures in which like reference numerals refer to like elements throughout and which:

FIG. 1 shows a side sectional view of a distal end of an imaging guidewire.

FIG. 2 shows a block diagram of a system controller.

FIG. 3 shows a block diagram of another embodiment of a system controller.

FIG. 4a shows a side view of a permanent magnet comprising a combination of multiple disc magnets

FIG. 4b shows a top view of a permanent magnet comprising a combination of multiple disc magnets.

FIG. 5 is a graph of magnetic field as a function of distance S for various combinations of disc magnets.

FIG. 6 shows the imaging guidewire introduced into a vascular cavity of a patient

FIG. 7 shows the imaging guidewire in the left anterior descending coronary artery

FIG. 8 shows a drive unit to the side of the guidewire.

FIG. 9 shows first and second orthogonal coils of wire adapted to generate a rotating magnetic field.

FIG. 10 shows a side sectional view of an imaging guidewire operated by first and second electromagnets.

FIG. 11 shows a side sectional view of an imaging guidewire having a relatively long flexible tubular housing adapted to provide a linear or curved path for slug movement.

FIG. 12 shows a representation of the imaging guidewire in a rectangular box useful in describing a determination of scanning position in three dimensions.

FIG. 13 shows a block diagram of a system arranged to determine scanning position of the imaging guidewire in three dimensions.

FIG. 14 shows a side sectional view of a distal end of a catheter arranged according to the invention.

FIG. 15 shows a proximal end of the catheter shown in FIG. 14.

### DETAILED DESCRIPTION OF THE INVENTION

This invention provides an apparatus and method for imaging tissues from inside a patient's body by utilizing a rotating magnetic field generated outside of the patient's body to cause a substantially synchronous rotation of an ultrasonic signal inside the patient's body.

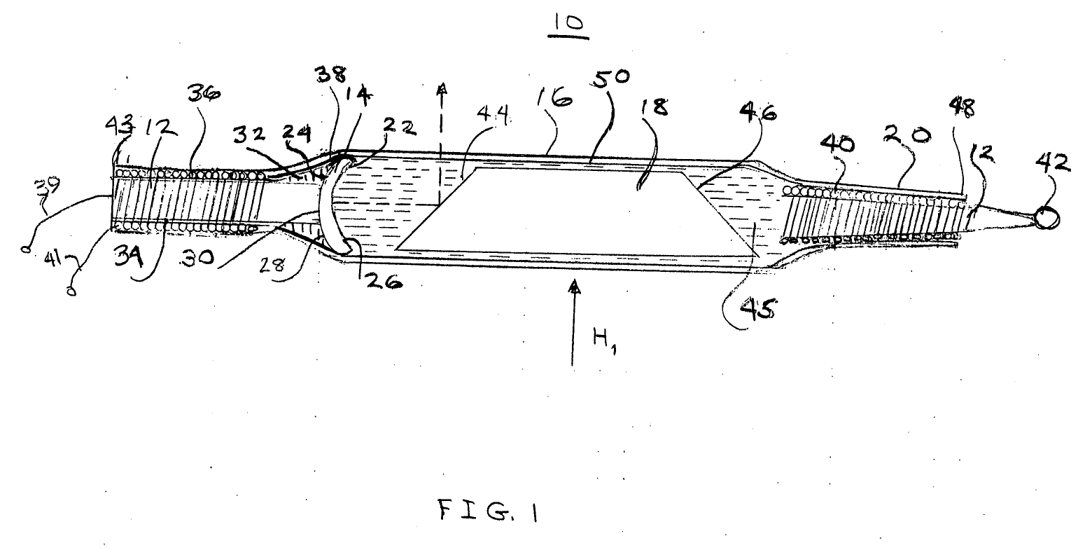

Referring to FIG. 1, there is shown a side sectional view of a catheter or distal end of an imaging guidewire 10 including a central core wire 12, a piezoelectric transducer 14, a tubular housing or sleeve 16, a permanently magnetized cylindrical slug 18, and a flexible tip guide 20. The slug 18 may be formed from a rare earth magnetic material such as neodymium iron boron or samarium cobalt or from a suitable ferrite material. The magnetization of slug 18, represented by the magnetic field vector H₁, is substantially orthogonal to the longitudinal axis of slug 18. A metal cladding 17 over slug 18 is intended to enhance acoustic reflection and minimize corrosion of the rare earth material. The piezoelectric transducer 14 is formed from suitable material such as lead zirconate titanate (PZT) with first and second opposed surfaces 22, 24 covered by metallic conductive films which serve as electrodes 26, 28. The piezoelectric transducer 14 may have an operating range of 5 to 50 megahertz and may have either a substantially flat or a concave shaped first surface or electrode 26 as shown in FIG. 1, for directing ultrasonic energy. The second surface or electrode 28 is mechanically and electrically connected to the end 30 of the central core wire 12 by a conductive epoxy 32. Heat shrinkable tubing 34 is shrunk tightly over the central core wire 12 and conductive epoxy 32 against the second electrode 28. The tubing 34 is intended to electrically insulate the central core wire 12 and second electrode 28 from a second wire 36 wound around the central core wire 12 on top of the tubing 34. A thin conductive film 38, such as silver paint, extends from the first electrode 26 to an end of the second wire 36. It will be understood that signals can be transmitted to and from the electrodes 26, 28 of the piezoelectric transducer 14 via leads 39 and 41 respectively connected to the central core wire 12 and the second wire 36. In addition, the combination of a central core wire 12 around which is wound a second wire 36 yields the proper flexibility needed to navigate within a vascular cavity.

The flexible tip guide 20 includes a tapered section of a central core wire 12 around which is wound a second wire 40. The tip guide 20 may be terminated in a ball 42 useful in determining position of the guide wire in a vascular cavity. The purpose of a tapering down the diameter of the central core wire 12 in the direction of the terminating ball 42 is to provide the guidewire 10 with greater flexibility while navigating a vascular cavity and to prevent the wire from stabbing and injuring the vascular cavity.

The tubular sleeve 16 can be formed from any suitable material transparent to ultrasonic signals. Examples of suitable flexible material are heat shrinkable plastic or Teflon, a Trademark, tubing or polyurethane tubing. The sleeve 16 can also be fabricated from relatively non-flexible material such as perforated stainless steel tubing covered by a relatively thin ultrasound transparent membrane. An end 43 of the tubular sleeve 16 is slipped over the piezoelectric transducer 14 and a section of the central core wire 12 and second wire 36. The magnetized slug 18 is disposed within the tubular sleeve 16 with one end 44 of the slug 18 beveled at an angle of 45 degrees, opposite the concave electrode 26 of the piezoelectric transducer 14. The inside diameter of the tubular sleeve 16 and the diameter of the slug 18 are selected so that the slug 18 may freely rotate about its longitudinal axis. The other end 46 of the slug 18 may also be beveled at an angle of 45 degrees to minimize wobble while the slug 18 is rotating.

The other end 48 of the tubular sleeve 16 is slipped over an end section of the tip guide 20. The tubular sleeve 16 is subjected to heat and shrunk tightly onto the tip guide 20, and section of the central core wire 12 and second wire 36. A sterile liquid 45, such as saline, is pressure-fed under the end 48 of the tubular sleeve 16 and into the chamber 50 containing the slug 18 prior to insertion of the guidewire 10 into a patient's body. The liquid 45 may also be sealed within sleeve 16 at the time of fabrication of guidewire 10. The sterile liquid 45 provides a near frictionless water bearing for rotation of the slug 18 with minimal drag and minimal static and kinetic friction.

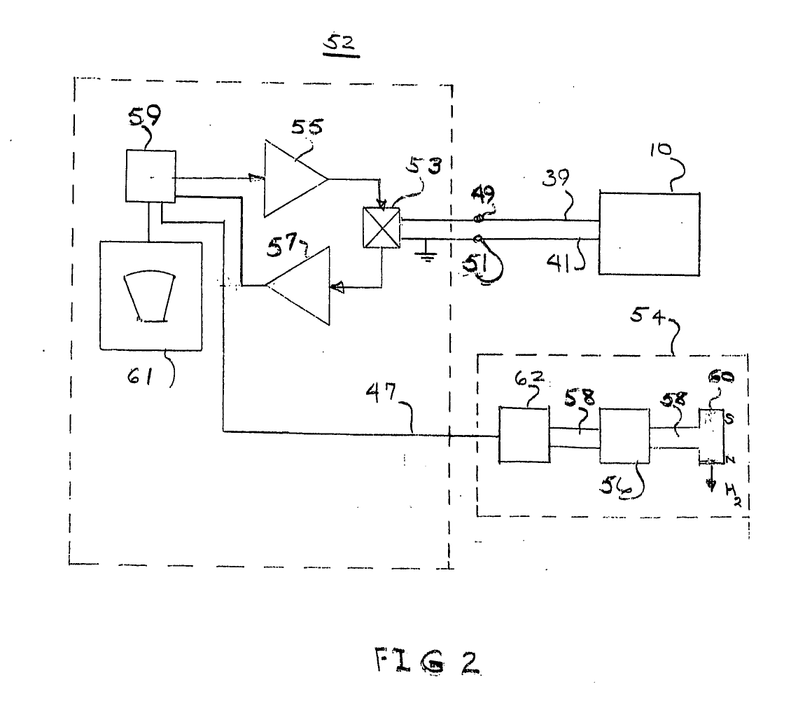

Referring to FIG. 2, there is shown a block diagram of a system controller 52 connected to a drive unit 54. The system controller 52 includes a transmitter 55 for transmitting electrical signals to the transducer 14 within the guidewire 10, a receiver 57 for receiving electrical signals from the transducer 14, a scan converter 59 for receiving signals from receiver 57 and drive unit 54 for processing and transmission of signals to display unit 61 and triggering the transmitter 55, a transmit/receive switch 53 for timing of electrical signals transmitted to the transducer 14 and reception of echo signals from transducer 14. The central core wire 12 and the second wire 36 are electrically coupled to terminals 49 and 51, respectively, of the system controller 52 via leads 39 and 41.

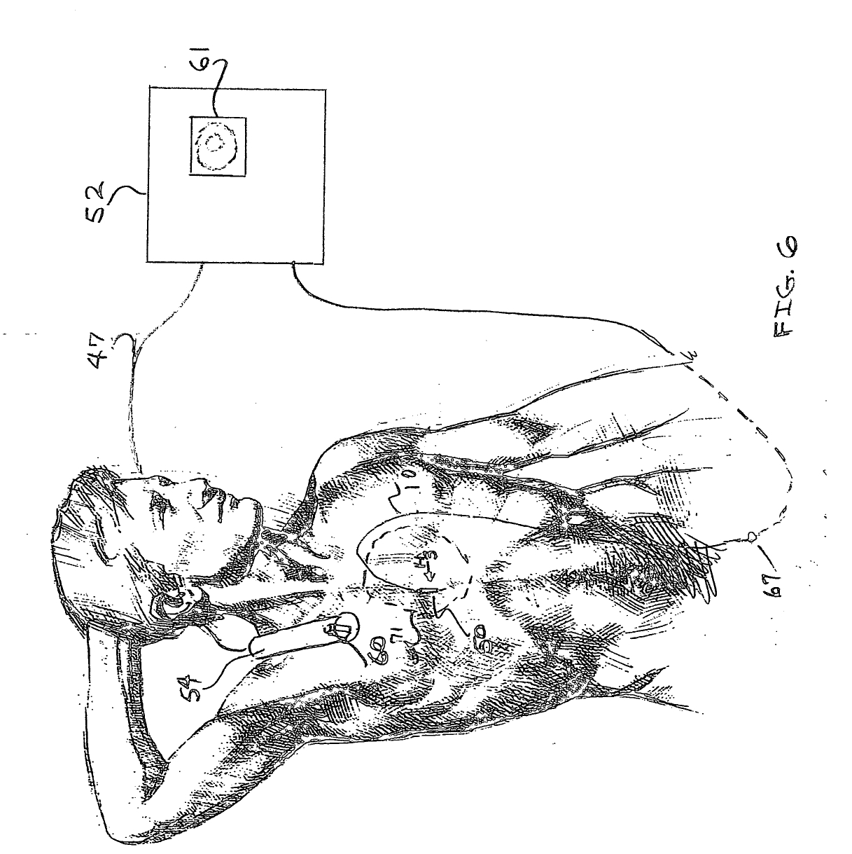

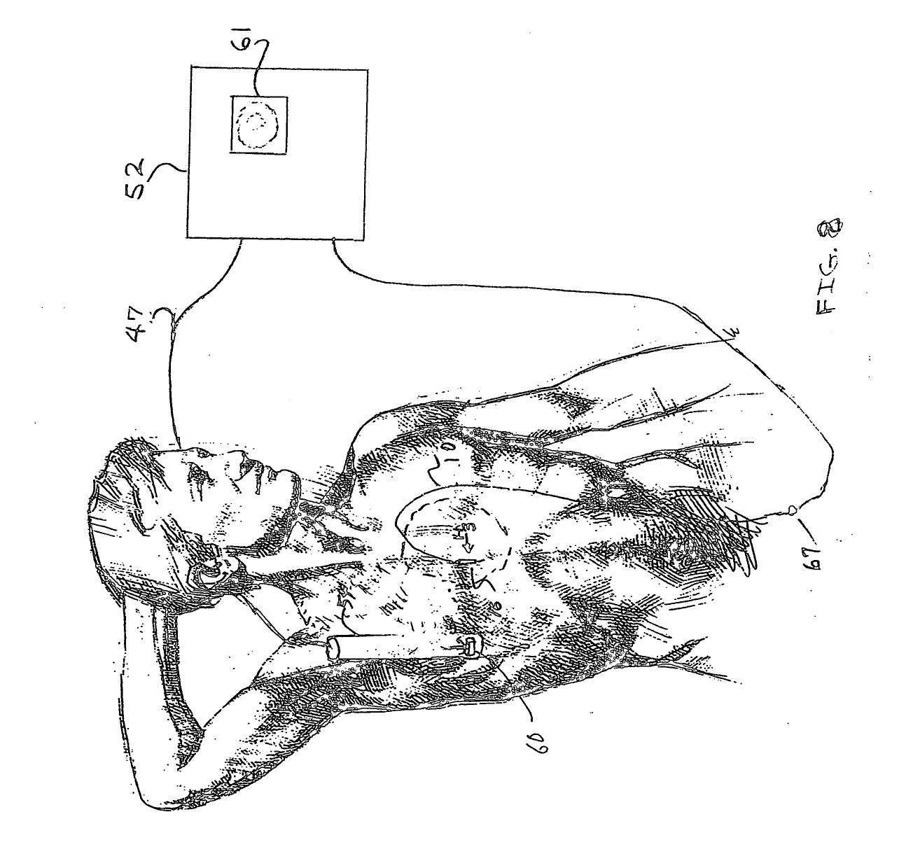

The drive unit 54 includes a motor 56 arranged to rotate a shaft 58 having a permanent magnet 60 attached at one end and an attached shaft encoder 62 for indicating angular displacement of the shaft 58. The permanent magnet 60 is magnetized in the direction shown by the vector H₂ and is intended to provide a magnetic field represented by the vector H₃, shown in FIG. 6 and FIG. 8, which rotates as the shaft 58 is rotated. The drive unit 54 is electrically connected to the system controller 52 and scan converter 59 via a flexible signal cable 47 so as to provide a signal indicating the angular position of magnet 60 and a corresponding angular position of slug 18 since rotation of magnet 60 and slug 18 are substantially synchronous and coincidental. Thus, signals indicating the angular position of slug 18 are coupled to scan converter 59 for further processing and transmission of signals to display unit 61. The signals received by display unit 61 are indicative of the condition of tissue scanned by imaging guidewire 10 and such signals are visually displayed by display unit 61.

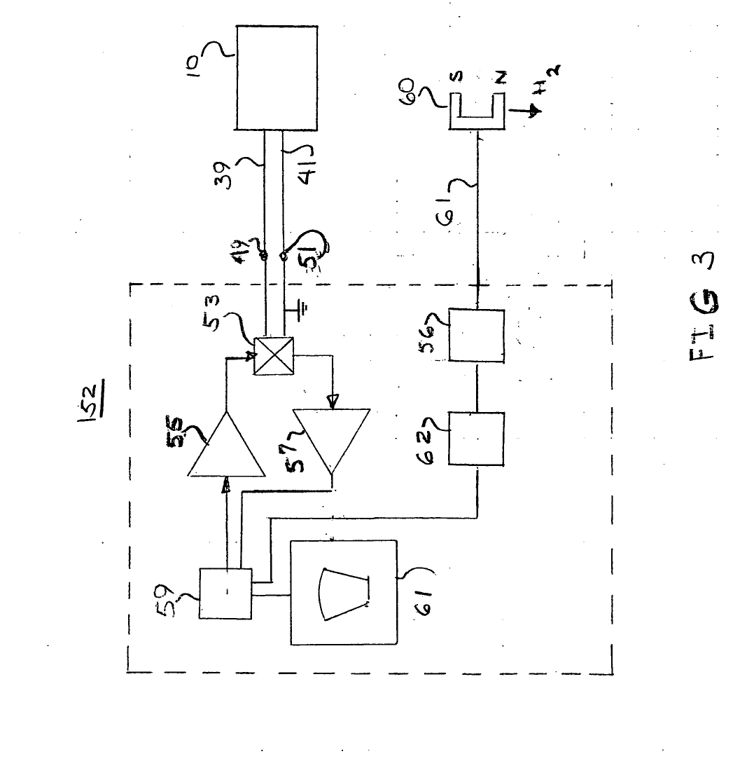

Referring to FIG. 3, there is shown a block diagram of another embodiment of a system controller 152 having shaft encoder 62 and motor 56 included as operating elements of system controller 152. An example of system controller 152 is the Galaxy TMZ IVUS Imaging System manufactured by Boston Scientific, Inc., Natick, Mass.. A flexible shaft 61 has one end coupled to motor 56 and another end attached to a permanent magnet 60.

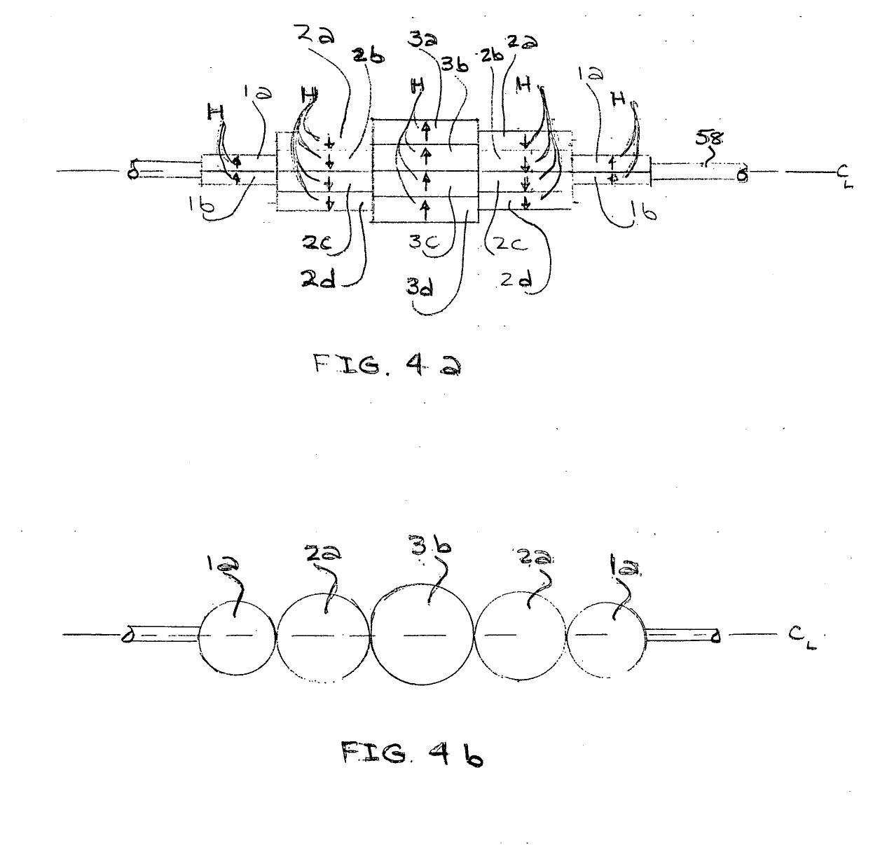

Referring to FIG. 4a and FIG. 4b, there is shown a side and top view of a more complex permanent magnet 60. The advantages of the complex permanent magnet 60 will become apparent later. The complex magnet 60 has a combination of sixteen individual permanent disc magnets of three different sizes attached to a rotatable shaft 58. A stack of two relatively small magnets 1a, 1b are arranged on shaft 58 at the beginning and end of magnet 60. First and second stacks of four magnets 2a, 2b, 2c, 2d are arranged on shaft 58 so that each stack is between a stack of two relatively small magnets 1a, 1b and a stack of four relatively large magnets 3a, 3b, 3c, 3d. The magnetic strength of the disc magnets is approximately proportional to the physical size of the magnets. The magnet stacks may be arranged so that the magnetic field vectors, H, for each magnetic stack alternate in direction providing five magnetic field direction alternations or in effect five composite magnets. It will be understood that a combination of twelve individual permanent disc magnets, not shown, of two different sizes forming three stacks with each stack having four magnets with magnetic field vectors alternating in direction is also a viable design.

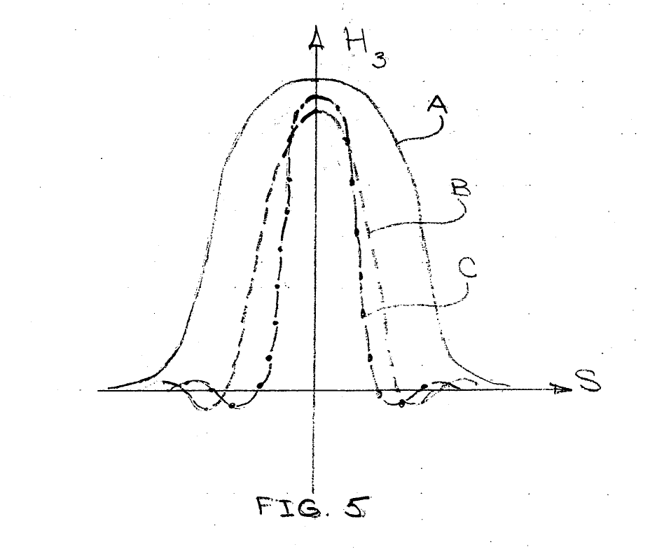

Referring to FIG. 5, there is shown a graph of magnetic field strength as a function of distance, S, along the length of sleeve 16 for a single magnet, curve A, three magnets, curve B, and five magnets, curve C. The combination of five magnets, curve C, provides a magnetic field strength or magnetic field gradient that changes in magnitude more rapidly as a function of distance along the sleeve longitudinal axis. As discussed below, it is sometimes desirable to move slug 18 within sleeve 16 along the longitudinal axis of sleeve 16 while simultaneously rotating slug 18 about its longitudinal axis. A permanent magnet 60 having a relatively high magnetic field gradient is particularly suited for moving slug 18 within sleeve 16 as the magnet 60 is moved in a direction substantially parallel to the longitudinal axis of slug 18.

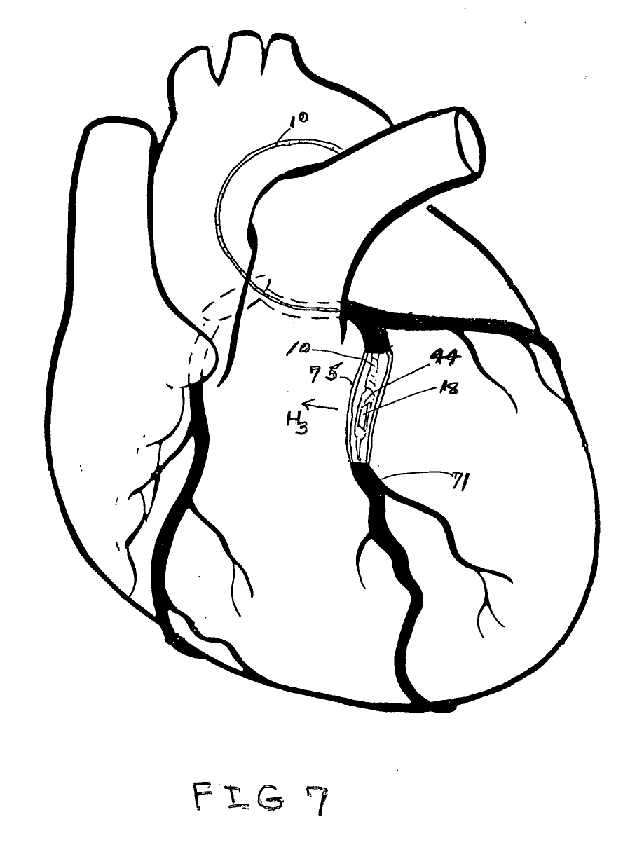

Referring to FIG. 6, there is shown the distal end of the imaging guidewire 10 introduced into a vascular cavity such as the femoral artery in the groin area 67 of a patient. X-ray fluoroscopy can be used to visually display the progress of the guidewire 10 in the vascular cavity and into the left anterior descending artery 71 shown in FIG. 7. The permanent magnet 60 within control unit 54 is located outside of sleeve 16 and outside the patient's body in the vicinity of the distal end of guidewire 10. Control unit 54 can be positioned near the neck of a patient as shown in FIG. 6, or to the side of guidewire 10 as shown in FIG. 8. The location of control unit 54 as in FIG. 8 is better suited to induce movement of slug 18 along the longitudinal axis of sleeve 16.

Preferably, the longitudinal axis of the motor shaft 58 (shown in FIG. 2) is substantially parallel to the longitudinal axis of the slug 18 so that the direction of the magnetic field (represented by vector H₃) provided by the permanent magnet 60 is in a plane substantially orthogonal to the longitudinal axis of the slug 18. Thus, angular displacement or rotation of the permanent magnet 60 will now cause a substantially synchronous rotation of the slug 18. The rotating slug end 44 is able to reflect and cause an impinging ultrasonic signal to traverse the arterial wall 75 (shown in FIG. 7) and be swept radially from 0 to 360 degrees about the slug's longitudinal axis. Ultrasonic signals reflected by tissue surrounding the guidewire 10 are received by the slug end 44 and directed toward the first electrode 26 of the piezoelectric transducer 14 for conversion to electrical signals which are transmitted to the system controller 52 via the central core wire 12 and second wire 36. The system controller 52 is adapted to act in response to such transducer generated signals to provide a visual image on the display unit 61 indicative of condition of tissue surrounding the guidewire 10.

The slug 18 will move linearly along the sleeve longitudinal axis from a first position to a second position within the sleeve 16 when the permanent magnet 60 within control unit 54 is manually moved along a path substantially parallel to the longitudinal axis of sleeve 16, whereby signals generated by transducer 14 would provide a visual image on display unit 61 indicative of the condition of the tissue surrounding guidewire 10 at the second position of slug 18.

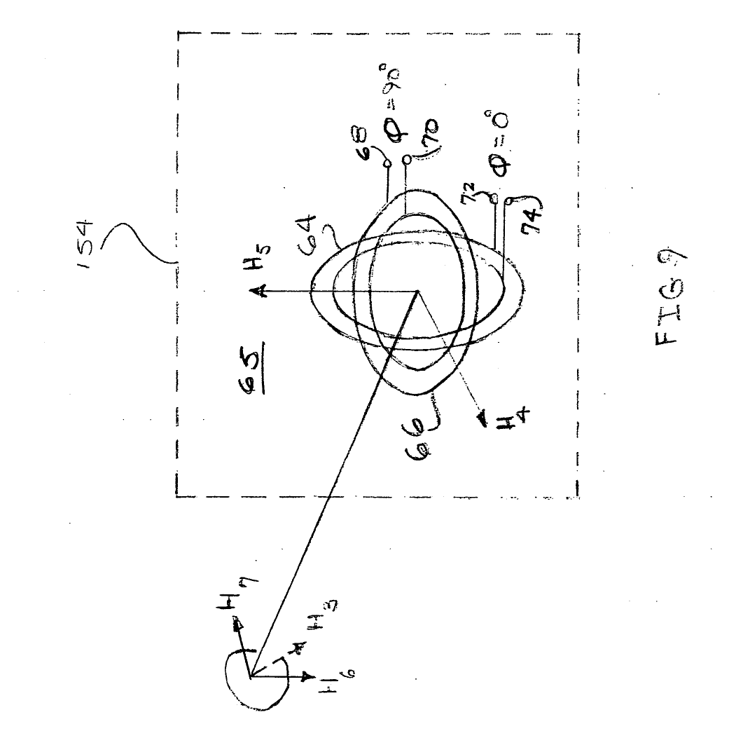

Referring to FIG. 9, there is shown a schematic of an embodiment of a drive unit 154 comprising an electromagnet 65 having first 64 and second 66 orthogonal coils of wire. An electrical power source (not shown) supplies equal amplitude AC current 90 degrees out of phase to the input terminals 68, 70, and at 0 degrees to terminals 72 and 74 of the coils, causing the coils to generate magnetic fields with directions represented by orthogonal vectors H₄ and H₅ which in turn produce magnetic fields represented by orthogonal vectors H₆ and H₇ and a rotating magnetic field represented by vector H₃. The magnitude of the rotating magnetic field H₃ may be adjusted as a function of time by changing the magnitude of the A C current. It will be understood that the coils 64, 66 may be used as an alternative to the rotating permanent magnet 60 as means for generating a rotating magnetic field for inducing rotation of the slug 18. Thus, it will be understood that in operation, the drive unit 154 is intended to be located outside of the sleeve 16 and outside of the patient's body in the vicinity of the distal end of guidewire 10.

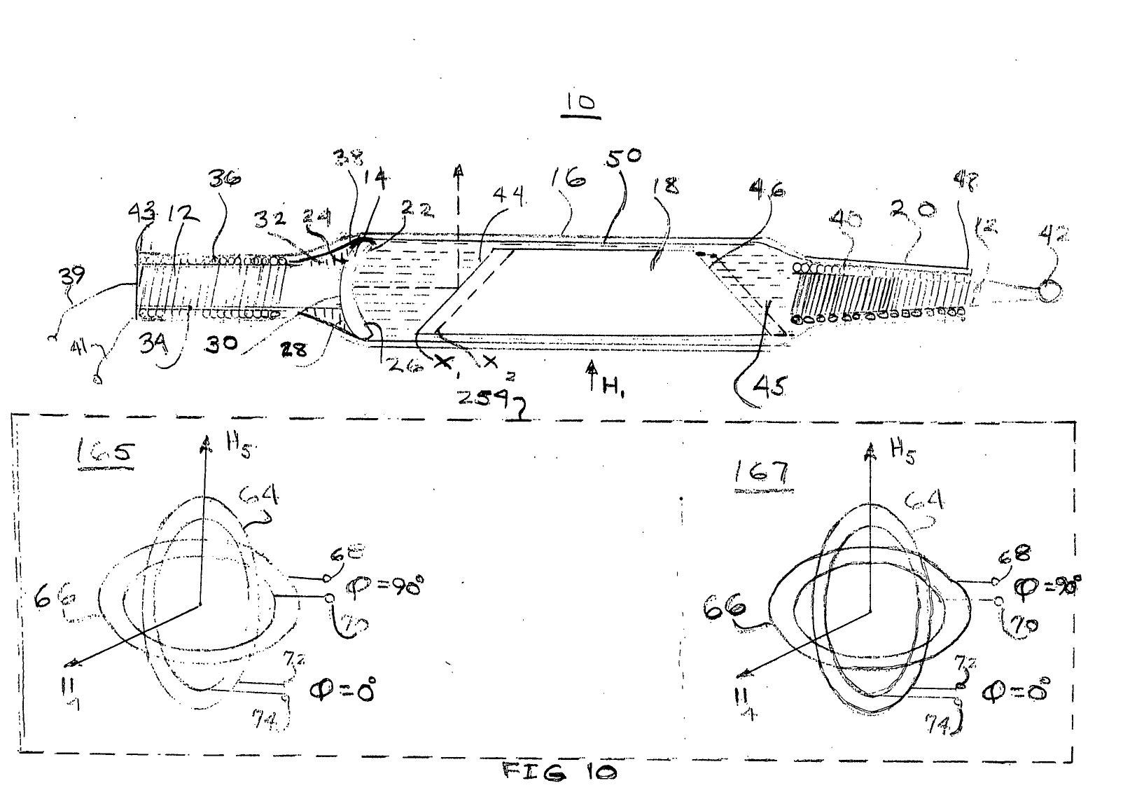

Referring to FIG. 10, there is shown guidewire 10 operated by a drive unit 254 comprising first 165 and second 167 electromagnets. The first electromagnet 165 is positioned near end 43 of sleeve 16 and the second electromagnet 167 is positioned near ball 42 of guidewire 10. Each of the electromagnets 165, 167 is operated by a separate electrical input signal to produce separate magnetic fields in the vicinity of the slug 18. The magnetic fields produced by the electromagnets 165, 167 are substantially parallel and rotating in substantially the same direction so that the electromagnets 165, 167 assist each other in causing rotation of the slug 18. The electromagnets 165, 167 are positioned outside of sleeve 16 and outside of the patient's body but relative to the position of the slug 18 so that the slug 18 may be induced by a magnetic force to move along the length of sleeve 16 as well as rotationally about the slug longitudinal axis. For example, if each of the electromagnets 165, 167 are located near different ends of slug 18 and the signal to the first electromagnet 165 is turned off while the input signal to the second electromagnet 167 is turned on, the gradient in magnetic field strength will induce the slug 18 to move within the sleeve 16 toward the second electromagnet 167. The slug 18 will move from position Xi to position X₂ since the slug 18 is drawn to the region of higher magnetic field while simultaneously rotating about the slug longitudinal axis. The motion is much like a threaded screw turning in a threaded hole. If the input signal to the first electromagnet 165 is turned on while the input signal to the second electromagnet 167 is turned off, then the movement of the slug 18 within the sleeve 16 is reversed. Alternatively, the phase of the input current signals to the electromagnets 165, 167 may be adjusted, as known in the art, so that the electromagnets 165, 167 produce magnetic fields that are unequal in magnitude and opposite in direction but still rotating in the same sense and causing rotation of slug 18. It will be apparent that, for example, slug 18 may be repelled by the magnetic field produced by electromagnet 165 and attracted by the magnetic field produced by electromagnet 167. The combination of the force of repulsion provided by the magnetic field produced by the electromagnet 165 and the force of attraction provided by the magnetic field produced by electromagnet 167 cause slug 18 to move within sleeve 16 along the sleeve longitudinal axis in the direction of electromagnet 167. The direction of slug 18 movement within sleeve 16 along the sleeve longitudinal axis may be reversed by reversing the phase of the current signals to the electromagnets 165, 167 so that slug 18 is repelled by the magnetic field produced by electromagnet 167 and attracted by the magnetic field produced by electromagnet 165.

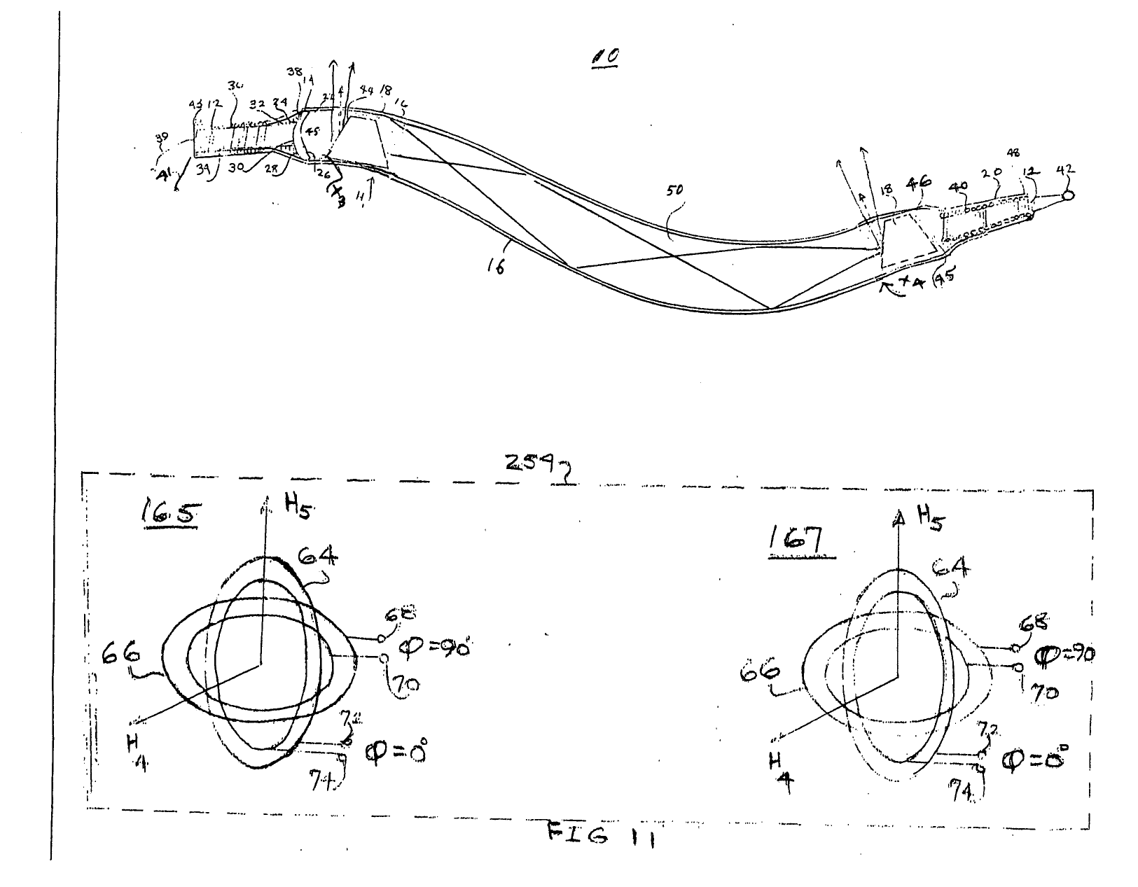

Referring to FIG. 11, there is shown a catheter or guidewire 10 having sleeve 16 formed from flexible material adapted to function as an acoustic waveguide with internal reflections permitting propagation of acoustic signals generated by the piezoelectric transducer 14 and transmitted to and from slug 18. It is sometimes desirable to move slug 18 along a curved path within sleeve 16 from a first position to a second position along the sleeve longitudinal axis while simultaneously rotating about the slug longitudinal axis while catheter or guidewire 10 remains stationary. For this reason, the length of sleeve 16 may be relatively long to allow the slug 18 to follow the curvature of the vascular cavity when catheter or guidewire 10 is operated by drive unit 254 as described in connection with FIG. 10. The slug 18 is able to move a relatively long distance within the sleeve 16 from position X₃ to position X₄ in the event the catheter or guidewire 10 is positioned in a curved portion of a vascular cavity.

It will be apparent to one skilled in the art that by adjusting the magnitude of the input current to each electromagnet 165, 167, the slug 18 can be scanned or moved back and forth along a curved path over the length of the sleeve 16 while the guidewire 10 is stationary. The specific position of slug 18 along the sleeve longitudinal axis can be determined from the ultrasound signal timing resulting from the partial reflection of the ultrasound pulse as it passes through and exits sleeve 16. The slug 18 can also be made to move from position X₃ to position X manually with the use of the more complex permanent magnet 60 shown in FIG. 4a and FIG. 4b. This is accomplished by moving the drive unit 54 shown in FIG. 8 in a direction substantially parallel to the axis of rotation of permanent magnet 60. The distance, S, that slug 18 is moved along the longitudinal axis of sleeve 16 is determined from equation 1:
S=υ×τ/2
where υ is the velocity of the ultrasound signal transmitted through liquid 45, and τ is the round trip time for an ultrasound signal to be transmitted from transducer 14 to a surface of sleeve 16 and reflected back to transducer 14. The data taken during motion of slug 18 from X₃ to X₄ can be stored and combined to form an enhanced image through synthetic aperture imaging techniques described in a text entitled “Acoustic Wave Devices, Imaging and Analog Signal Processing” by G. S. Kino published by Prentice-Hall Inc., Englewood Cliffs, N.J.

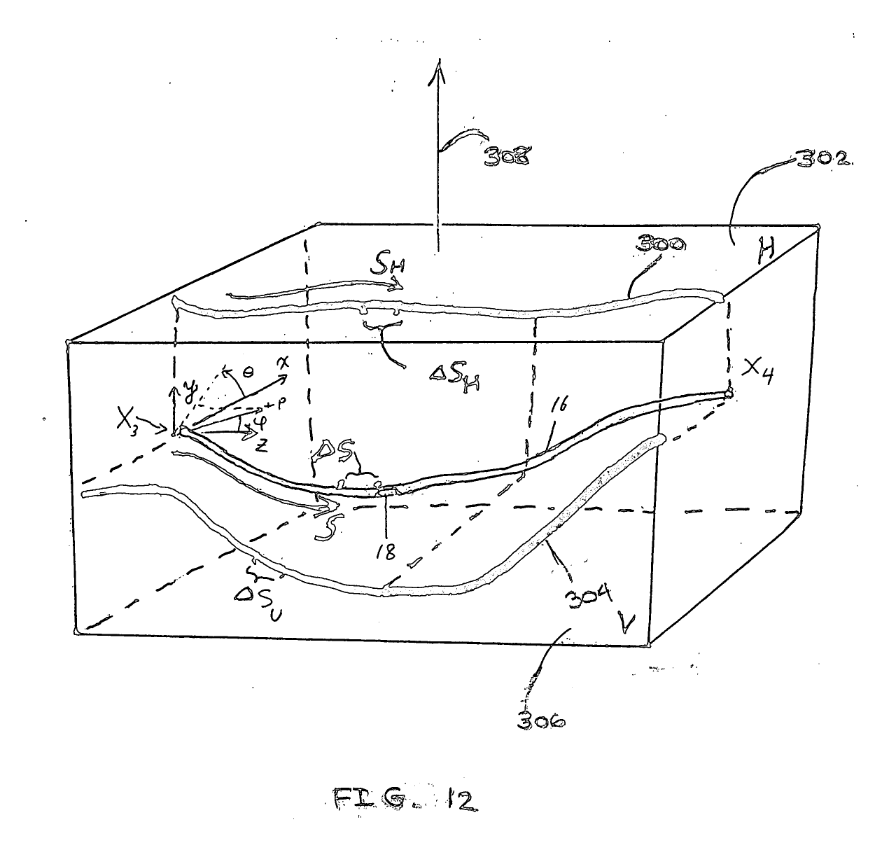

Referring to FIG. 12, there is shown a representation of a sleeve 16 of catheter or guidewire 10 positioned in a curved portion of a vascular cavity. The slug 18 is induced by an exterior magnetic field produced by drive unit 254 or by manual motion of drive unit 54 to move from position X₃ to position X₄ along a path, S, within sleeve 16 and following the curvature of sleeve 16. For convenience, the sleeve 16 is shown as contained within an imaginary rectangular box useful in illustrating a projection 300 of the curvature of sleeve 16 onto a horizontal plane 302 and a projection 304 of the curvature of sleeve 16 onto an orthogonal vertical plane 306. As the slug 18 is moved a relatively small distance, ΔS, there are corresponding changes ΔS_{υ} and ΔS_{H} in the projections 300, 304 in the horizontal 302 plane and the vertical 306 plane The changes in ΔS_{υ} and ΔS_{H} are related by the equation 2:
ΔS²=ΔS_{H}²+ΔS_{υ}²
where ΔS is determined by a change in the round trip timing of the leading edge of an ultrasound pulse traveling from transducer 14 to the surface of sleeve 16 and reflected back to transducer 14 The partial reflection of the ultrasound pulse reflected by sleeve 16 is transmitted by transducer 14 back to receiver 57 for determination of the quantity ΔS.

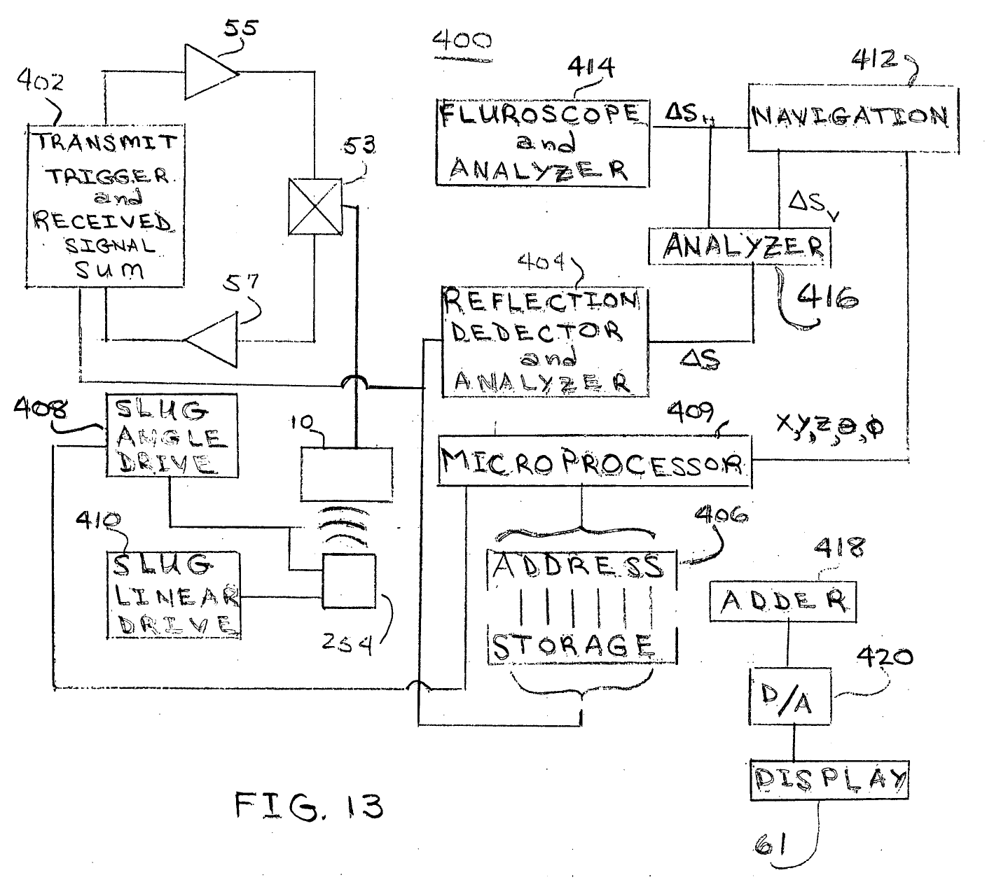

A fluoroscope and analyzer 414, shown in FIG. 13, is arranged adjacent to 302 to detect a beam 308 and therein detect the change ASH of the position of slug 18 as projected on horizontal plane 302. It will be apparent that the quantity ΔS_{υ} can be determined from equation 2 and used to establish the next increment of the vertical projection of the movement of slug 18. The angular positions φ and θ of the longitudinal axis of slug 18 relative to the x, y, and z axis can be determined from the sequence of the quantities ΔS, ΔS_{υ}, and ΔS_{H}. Thus, after one complete traverse of slug 18, the projections 304, 300 of slug 18 positions onto the vertical 306 and horizontal 302 planes can be established and the position of slug 18 in three dimensions on the x, y, and z axis and the angular positions φ and θ of slug 18 at any point within sleeve 16 can be determined as well as the condition of tissue being scanned by guidewire 10 at such point. The position of slug 18 along the x, y, and z axis is known relative to the position X₃

Referring to FIG. 13, there is shown a block diagram of an imaging system 400. The system 400 includes a transmitter 55 for transmitting electrical signals to the transducer 14 within the catheter or guidewire 10, a receiver 57 for receiving electrical signals from the transducer 14, a transmit trigger and received signal summer 402 for receiving and processing signals from receiver 57 for transmission to reflection detector 404 and to an address and storage unit 406. Slug angle drive unit 408 and slug linear drive unit 410 are coupled to drive unit 254 and arranged to operate drive unit 254 located outside of a patient's body to generate a magnetic field for causing slug 18 to rotate about its longitudinal axis and move along its longitudinal axis.

The slug angle drive unit 408 is coupled to a microprocessor 409 receiving information from slug angle drive unit 408 concerning slug angle rotation and information from a navigation unit 412 providing signals indicative of positions of slug 18 along an x, y, and z axis and angular positions θ, φ of slug 18 relative to the x, y, and z axis. A fluoroscope and analyzer 414 provides signals indicative of the change in slug position ΔS_{H} in the horizontal plane 302 to the navigation unit 412 and analyzer 416. The reflection detector 404 provides signals to the analyzer 416 that are indicative of a change in slug 18 position ΔS along the sleeve longitudinal axis as determined by a change in round trip timing of the ultrasound pulse from the transducer 14 to the surface of sleeve 16 and back to transducer 14. The analyzer 416 computes the change in slug position ΔS_{υ} in the vertical plane 306 and transmits signals indicative of ΔS_{υ} to the navigation unit 412 which in turn provides to the microprocessor 409 signals indicative of positions of slug 16 along the x, y, and z axis and angular positions θ, φ, of slug 18 relative to the x, y, and z axis. The microprocessor 409 processes signals from the slug angle driver 408 and navigation unit 412 and transmits signals to an address and storage device 406 for storing in memory the address and data information of all positions and rotations of slug 18 in three dimensions. The data base information is coupled to an adder 418 and digital-to-analog converter 420 for further processing to form a synthetic aperture image on display unit 61.

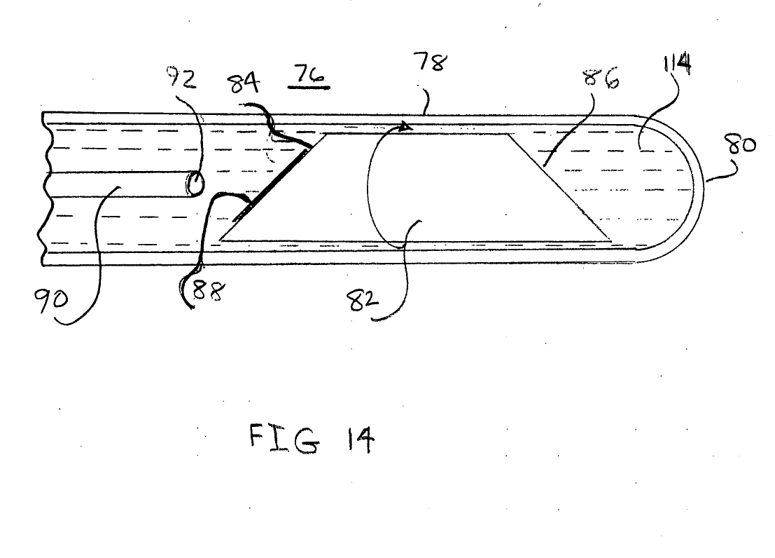

Referring to FIG. 14, there is shown a side sectional view of a distal end of a catheter 76 adapted for insertion into a vascular cavity of a human body. The catheter 76 comprises an elongated tubular body 78 having a generally rounded tip 80. The tubular body 78 is formed from a material substantially transparent to ultrasonic signals and suitable for inserting into a vascular cavity with minimal friction. A permanently magnetized cylindrical slug 82 is disposed within the tubular body 78 near the tip 80. The slug 82 has one 84 or both ends 84, 86 beveled at an angle of 45 degrees and is adapted to rotate freely about its longitudinal axis within the tubular body 78. At least one 84 of the beveled ends 84, 86 may have a reflective surface 88 comprising a smooth coating of acoustic reflective material. An acoustic waveguide 90 is coaxially disposed within the tubular body 78. An end 92 of the acoustic waveguide 90 is positioned opposite the reflective surface 88 of the slug 82.

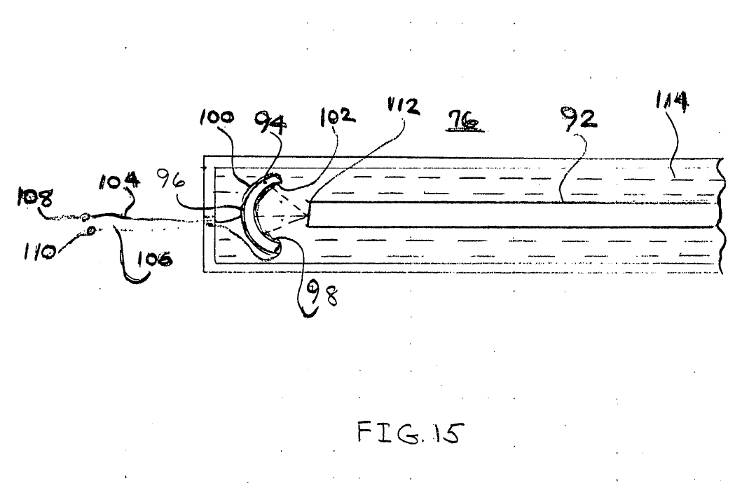

Referring to FIG. 15, there is shown a proximal end of the catheter 76. The proximal end houses a spherically shaped piezoelectric transducer 94 having convex 96 and concave 98 surfaces. It is known that the radius of curvature of the transducer 94 determines the focal point of an ultrasonic signal generated by the transducer 94. The convex 96 and concave 98 surfaces of the piezoelectric transducer 94 are each metalized with a conductive film to provide electrodes 100, 102 which are electrically connected to wires 104, 106 extending rearward from the tubular body 78. The wires 104, 106 have terminals 108, 110 for receiving electrical signals used to drive the transducer 94 to generate acoustic or ultrasonic signals.

The proximate end 112 of the acoustic waveguide 90 is positioned at the focal point of the ultrasonic signals generated by the transducer 94 whereby ultrasonic signals may be transmitted to the distal end 92 of the acoustic waveguide 90. The acoustic signals emitted from the distal end 92 of the acoustic waveguide 90 are reflected off the reflective surface 88 of the slug 82 and transmitted through the tubular body 78. The end 84 of the slug 82 is beveled at 45 degrees to cause the acoustic signals emitted from the waveguide 90 to be reflected at an angle substantially transverse or 90 degrees to the longitudinal axis of the slug 82.

An acoustical coupling fluid 114 is contained within the tubular body 78 filling the space between the transducer 94 and acoustic waveguide 90 and the space between the slug 82 and tubular body 78. The fluid 114 is intended to enhance a coupling of acoustic signals generated by the transducer 94 into the proximate end 112 of the acoustic waveguide 90 and between the distal end 92 of the acoustic waveguide 90 and the reflective surface 88 on the slug 82. The fluid also acts as a lubricant or near frictionless bearing between a surface of the rotating slug 82 and an inner surface of the tubular body 78.

In operation, the distal end of the catheter 76 is inserted into a vascular cavity of a patient. A prior art guidewire (not shown) may be attached to the catheter 76 to enable an operator to steer the distal end of the catheter 76 to a desired location. An example of a suitable guidewire and method of attachment is described in U.S. Pat. No. 5,507,294. A rotating magnetic field generated by a drive unit located outside of the patient's body is used to induce rotation of the slug 82. Examples of such a drive unit and its operation is the motor driven rotating permanent magnet 60 shown and described in connection with FIG. 2, or the electromagnet 65 shown and described in connection with FIG. 5, or an arrangement of first 165 and second 167 electromagnets shown and described in connection with FIG. 6a Electrical signals from the system controller 52 shown and described in FIG. 2 are coupled to the terminals 108, 110 for driving the transducer 94 to generate acoustic or ultrasonic signals. Acoustic signals emitted from the waveguide distal end 92 are reflected by the reflective surface 88 on the slug 82 at an angle substantially transverse to the longitudinal axis of the slug 82. The drive unit 54 or 154 or 254 for generating a magnetic field outside of the patient's body is operated to cause rotation of the slug 82 whereby the reflected acoustic signal may be swept or angularly displaced over any predetermined angle from 0 to 360 degrees. The slug 82 may also be linearly displaced or moved along a curved path within body 78 in the event a drive unit 254 including first 165 and second 167 electromagnets is used as shown and described in connection with FIG. 6a or FIG. 6b. Acoustic signals reflected by body tissue return along the same path traveled by acoustic signals generated by the transducer 94. The transducer 94 converts such tissue-reflected acoustic signals into corresponding electrical signals which provide image data or information on the contours of the body tissue. The system controller 52 receives such image data and rotation rate and angular displacement of the slug 82 and displays a two-dimensional image on a CRT or display unit 61 that is indicative of the condition of the body tissue at each angular position.

While this invention has been shown and described with reference to preferred embodiments hereof, it will be understood by those skilled in the art that various changes in form and details may be made therein without departing from the spirit and scope of the invention as defined by the appended claims.

What is claimed is:

1. In an imaging guidewire for imaging tissues from inside a patient's body cavity having a wall, the imaging guidewire having a distal end suitable for inserting inside the body cavity, the improvement comprising:
a length of substantially tubular housing having a portion that is substantially transparent to ultrasound, the housing being proximate to the distal end of the imaging guidewire;
a permanently magnetized cylindrical slug disposed within the housing, the slug having a longitudinal axis and at least one beveled end;
an ultrasonic beam transmitting means disposed within the housing for transmitting an ultrasonic beam toward the beveled end of the slug for reflecting the ultrasonic beam toward the housing and the wall of the body cavity; and
means for generating a rotating magnetic field from outside of the patient's body to cause substantially synchronous rotation of the slug substantially about the slug longitudinal axis and rotational movement of the ultrasonic beam for scanning the ultrasonic beam at the wall of the body cavity for imaging.

2. An imaging guidewire in accordance with claim 1, wherein the slug has first and second beveled ends.

3. An imaging guidewire in accordance with claim 1, wherein the slug end is beveled at substantially 45 degrees.

4. An imaging guidewire in accordance with claim 1, further including means for projecting an image of the slug onto a substantially planar surface.

5. An imaging guidewire in accordance with claim 1, wherein the means for generating a rotating magnetic field include an electromagnet.

6. An imaging guidewire in accordance with claim 1, wherein the means for generating a rotating magnetic field include a permanent magnet attached to one end of a shaft rotated by a motor.

7. An imaging guidewire in accordance with claim 1, further comprising a liquid contained within the housing for providing a liquid bearing around the slug.

8. An imaging guidewire in accordance with claim 1, further comprising first and second electromagnets positioned outside of the tubular housing for generating a rotating magnetic field to cause substantially synchronous rotation of the slug substantially about the slug longitudinal axis and rotational movement of the ultrasonic beam for scanning the ultrasonic beam at the wall of the body cavity and movement of the slug a predetermined distance along a selected path over the length of the tubular housing

9. An imaging guidewire in accordance with claim 1, wherein the means for generating a rotating magnetic field include a plurality of permanent magnets arranged with magnetic field vectors alternating in direction, the permanent magnets being attached to one end of a shaft rotated by a motor.

10. An imaging guidewire in accordance with claim 1, means for determining a period of time for a portion of the transmitted ultrasonic beam to travel from the ultrasonic beam transmitting means toward the housing and reflected by the housing back to the ultrasonic beam transmitting means.

11. An imaging guidewire in accordance with claim 1, wherein the slug is metal clad neodymium iron boron.

12. A method for imaging tissues from inside a patient's body cavity having a wall, comprising:
inserting into the body cavity a distal end of a catheter having a transducer positioned in a housing at the catheter's distal end;
generating an ultrasonic beam with the transducer;
directing the ultrasonic beam toward a beveled end of a magnetized cylindrical slug positioned in the housing for reflecting the ultrasonic beam toward the housing and the body cavity wall; and generating a rotating magnetic field outside of the patient's body cavity to cause rotation of the slug and rotational movement of the ultrasonic beam for scanning the ultrasonic beam at the body cavity wall for imaging.

13. A method according to claim 12 further comprising operating a motor to rotate a shaft having a permanent magnet at one end to generate a rotating magnetic field outside of the patient's body cavity.

14. A method according to claim 12 further comprising operating an electromagnet to generate a rotating magnetic field outside of the patient's body cavity.

15. A method according to claim 12 further comprising operating first and second electromagnets to generate a rotating magnetic field outside of the patient's body cavity to cause simultaneous rotational and axial movement of the slug within the tubular housing for scanning the ultrasonic beam at the body cavity wall for imaging.

16. A method according to claim 12 further comprising projecting an image of the slug onto a substantially planar surface.

17. A method according to claim 12 further comprising determining a period of time for a portion of the ultrasonic beam to be directed toward the housing and reflected by the housing back to the transducer.

18. A catheter for imaging tissues from inside a patient's body cavity having a wall comprising:
a length of substantially tubular housing having a portion substantially transparent to ultrasonic signals;
a magnetized cylindrical slug disposed within the tubular housing, the slug having a longitudinal axis and at least one beveled end;
an ultrasonic beam transmitting means disposed within the housing opposite the slug beveled end for directing an ultrasonic beam toward the slug beveled end; and
means for generating a rotating magnetic field outside of the housing to cause rotation of the slug about the slug longitudinal axis and rotational movement of an ultrasonic beam generated by the ultrasonic beam transmitting means and reflected by the slug beveled end for scanning of the ultrasonic beam at the body cavity wall for imaging.

19. A catheter in accordance with claim 18, wherein the slug end is beveled at 45 degrees.

20. A catheter in accordance with claim 18, wherein the ultrasonic beam transmitting means include a transducer opposite the slug beveled end.

21. A catheter in accordance with claim 18, wherein the ultrasonic beam transmitting means include an acoustic waveguide disposed within the housing between the transducer and slug beveled end.

22. A catheter in accordance with claim 18, wherein the means for generating a rotating magnetic field include an electromagnet.

23. A catheter in accordance with claim 18, wherein the means for generating a rotating magnetic field include a permanent magnet attached to one end of a shaft rotated by a motor.

24. A catheter in accordance with claim 18, further comprising drive means positioned outside of the tubular housing for generating a magnetic field to cause movement of the slug a predetermined distance along the length of tubular housing.

25. A catheter in accordance with claim 24, wherein said drive means comprise first and second electromagnets.

26. A catheter in accordance with claim 18, further comprising means for projecting an image of the slug onto a substantially planar surface.

27. A catheter in accordance with claim 18, further comprising means for determining a period of time for the transmitted ultrasonic beam to travel from the ultrasonic beam transmitting means to the housing and reflected by the housing back to the ultrasonic beam transmitting means.
